(19) United States
(12) Patent Application Publication
(10) Pub. No.: US 2013/0071890 A1
(43) Pub. Date: Mar. 21, 2013

(54) Production of PHA using Biogas as Feedstock and Power Source

(71) Applicants: Craig S. Criddle, Redwood City, CA (US); John R. Hart, Sacramento, CA (US); Wei-Min Wu, Okemos, MI (US); Eric R. Sundstrom, Palo Alto, CA (US); Margaret C. Morse, Menlo Park, CA (US); Sarah L. Billington, Palo Alto, CA (US); Katherine H. Rostkowski, Washington, DC (US); Curtis W. Frank, Cupertino, CA (US)

(72) Inventors: Craig S. Criddle, Redwood City, CA (US); John R. Hart, Sacramento, CA (US); Wei-Min Wu, Okemos, MI (US); Eric R. Sundstrom, Palo Alto, CA (US); Margaret C. Morse, Menlo Park, CA (US); Sarah L. Billington, Palo Alto, CA (US); Katherine H. Rostkowski, Washington, DC (US); Curtis W. Frank, Cupertino, CA (US)

(21) Appl. No.: 13/421,771

(22) Filed: Mar. 15, 2012

Related U.S. Application Data
(60) Provisional application No. 61/465,143, filed on Mar. 15, 2011.

(51) Int. Cl. C12P 7/62

(52) U.S. Cl. CPC C12P 7/625
USPC 435/135

(57) ABSTRACT

Methods for producing bioplastics from biogas include techniques for the production of PHB using a dirty biogas (e.g., methane from landfill, digester) as both a power source for the process and as feedstock. Biogas is split into two streams, one for energy to drive the process, another as feedstock. Advantageously, the techniques may be implemented off the power grid with no dependence upon agricultural products for feedstock.

### CROSS-REFERENCE TO RELATED APPLICATIONS

This application claims priority from U.S. Provisional Patent Application 61/465,143 filed Mar. 15, 2011, which is incorporated herein by reference.

### FIELD OF THE INVENTION

The present invention relates generally to methods for producing bioplastics from biogas. More specifically, the present invention provides techniques for the production of PHB using a dirty biogas (e.g., methane from landfill, digester) as both a power source for the process and as feedstock. Advantageously, the techniques may be implemented off the power grid with no dependence upon agricultural products for feedstock. Biogas is split into two streams, one for energy to drive the process, another as feedstock.

### BACKGROUND OF THE INVENTION

Current polymers and conventional recycling practices are not sustainable. Five of the “big six” polymers—high and low density polyethylene, polyvinyl chloride, polystyrene and polypropylene—can be reclaimed, but are typically downcycled in a single cycle to lower value products. The products themselves are persistent, so end-of-service disposal can be problematic, especially in space-constrained urban environments, and unintended consequences can result, including release of harmful chemicals into food, water, and air, and littering, with accumulation of debris patches over vast regions of the ocean. Renewable materials are needed that are economical and safe, and can substitute for petrochemical plastics in many applications. Renewable bioplastics and biocomposites are available, but their production currently relies upon the use of cultivated feedstock, such as corn, and large amounts of land, water, chemicals, and energy for growth, harvesting, transport, and processing of cultivated feedstock.

### SUMMARY OF THE INVENTION

To decrease costs, and to reduce organic waste entering landfills, bioplastics and biocomposites can be made from collected organic streams that are often perceived as “waste”, including low-value, limited-use plant biomass (e.g., yard waste); biorefinery residues; animal manure; municipal solid waste; food processing wastes; and bioproducts collected at end-of-life.

An attractive “waste” feedstock for production of renewable bioplastics is the biogas that is commonly produced at landfills, wastewater treatment plants, biorefineries, dairies, and food processing facilities. Biogas is a mixture of methane (50-60%) and carbon dioxide (40-50%). Landfills and large wastewater treatment plants produce thousands of tons of biogas per year. Co-location of a biorefinery at a biogas source can thus ensure a stable supply of virtually free feedstock of consistent quality. If not captured, methane is a potent greenhouse gas that will contribute significantly to climate change. If captured, its value depends on its purity. Clean biogas can be burned for energy. But low quality biogas may contain contaminants that require removal before energy can be recovered, such as hydrogen sulfide and siloxanes. In such cases, collected biogas is often flared. An advantage of the present invention is that unpurified biogas can be used as a feedstock for production of bioplastic.

One important organic waste stream is the organic fraction of municipal solid waste (MSW). There is already an infrastructure to collect MSW and bring it to landfills. In California, MSW passes through a sorting facility called a Materials Recovery Facility (MRF) prior to landfilling. At the MRF, metals, cans and bottles are removed for recycling. At the end of the process, what is left is called the MRF residue. This residue has a large percentage of cellulosic biomass. It can be converted to biogas methane in anaerobic digesters. About one third of the remaining material is lignin, a carbonaceous material that is not converted into biogas. The lignin can potentially be used as an additive in biopolymer products or burned to offset the energy demands of biomaterials synthesis.

This invention is a biorefinery that primarily produces bioplastic resins and biocomposites from waste feedstock, with biogas methane as a key feedstock and end product. The biodegradable portion of the waste stream can be converted into biogas. The remaining fraction of the waste or a portion of the biogas methane can be oxidized to supply the energy requirements for synthesis of bioplastic resins and fabrication of biocomposites. The result is a biorefinery for production of bioplastic resins and biocomposites that is sustainable economically and environmentally, with minimal reliance upon imported carbon and energy derived from fossil carbon feedstock.

This invention is a sustainable cradle-to-cradle biorefinery where organic waste streams are subject to anaerobic biodegradation to produce biogas methane, the biogas methane is used as feedstock for aerobic biosynthesis of biodegradable bioplastic resin and fabrication of bioplastic-containing biocomposites. At end-of-life, bioproducts made from the resin are converted back into the biogas methane feedstock. A fraction of the waste stream or of the biogas methane may be combusted to meet the energy demands of synthesis and fabrication, decreasing or eliminating the need for imported energy derived from fossil carbon. Management of biomaterials in this manner sequesters carbon, preserves limited landfill space, and decreases negative environmental impacts of bioplastics production.

### BRIEF DESCRIPTION OF THE DRAWINGS

FIG. 1. A flow diagram illustrating a process cycle including techniques of a preferred embodiment of the invention.

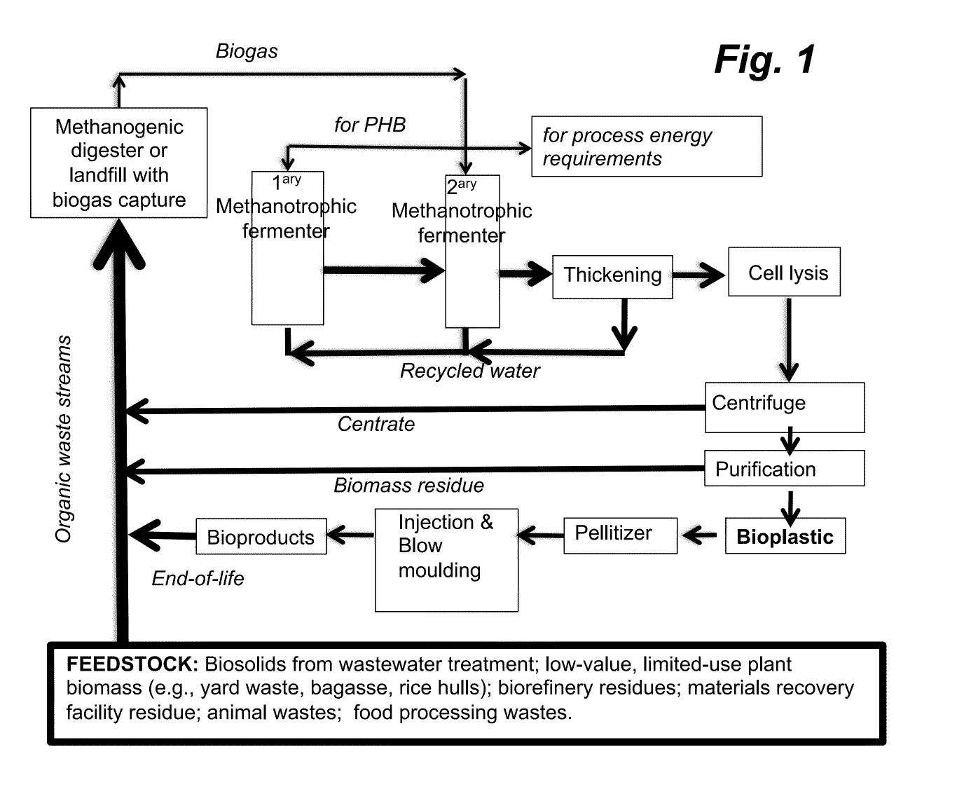

FIG. 2. Production of biogas from PHB resin (NODAX) incubated with anaerobic digester sludge at 30° C. and 37° C.

FIG. 3. Production of biogas from three biocomposites: PHB/Hemp, cellulose acetate/hemp, and soy bean oil/hemp.

### DETAILED DESCRIPTION

The invention is a biorefinery for sustainable biopolymer production. The term “sustainable” is here used to describe the environmental and economic benefits of the invention when compared to conventional methods that rely upon petrochemical feedstock or feedstock that is cultivated, harvested, and processed to produce building blocks for biopolymer production. Environmental benefits include enhanced carbon sequestration and decreased ocean acidification. Economic benefits accrue from the creation of local jobs tied to local waste feedstock. Because the feedstock is obtained from organic waste streams, landfill space is conserved, and bioplastic is produced using a feedstock and processes that do not depend upon imported sources of carbon and energy and are resistant to fluctuations in the price of food and energy. Additionally, sites of biogas methane production are commonly located near high density population centers, where plastics are commonly processed, thus decreasing transportation times and corresponding environmental impacts. Yet another benefit is distributed PHB production.

Many organic waste streams (including yard wastes, agricultural residues, forestry wastes, biorefinery residues, municipal solid waste, livestock wastes, food processing wastes, and bioproducts at end-of-life) are collected and converted into biogas in anaerobic digesters and at landfills. Within these anaerobic environments, self-assembled communities of anaerobic microorganisms convert the complex biopolymers that make up the biodegradable fraction of organic solids into soluble molecules, such as simple sugars, which are fermented into shorter-chain volatile fatty acids (formate, acetate, propionate, butyrate, lactate), carbon dioxide, and hydrogen, and subsequently degraded to produce biogas, a mixture of methane (40-70%) and carbon dioxide (30-60%).

Production of bioplastic from biogas requires a biogas feed system, a primary fermenter for growth of aerobic methanotrophic bacteria capable of biopolymer production and a secondary fermenter in which bioplastic production is induced in the presence of methane. In the primary fermenter, biogas methane, oxygen, and all of the required nutrients for growth are provided to enable rapid cell division. In the secondary fermentation, methane and oxygen are provided, but one or more of the remaining nutrients needed for growth are removed to induce bioplastic production. Bioplastic accumulates as granules inside the cells. The bioplastic-rich biomass is sent to a thickening device (belt press, dissolved air flotation device, etc.) to remove most of the water. In an alternative configuration, the biomass grown in the primary fermenter may be thickened prior to induction in the secondary fermenter.

Bioplastic production is followed by lysis of the cells to release the bioplastic granules from the cells. In the preferred embodiment, lysis is achieved without use of solvents or surfactants. Heating, sonic or electrical pulses, enzymes, or phage may be used to break the cells and release the granules from the cells. In the case of osmophilic methanotrophs, differences in osmotic pressure may be used to break the cells. The bioplastic is then separated from the remaining biomass and purified using one of several methods, including centrifugation to recover a biopolymer pellet, solvent extraction with solvent distillation and reuse, supercritical fluid extraction, and selective dissolution of residual biomass with sodium hypochlorite solutions. The biomass residuals are returned to the anaerobic digester for conversion into biogas.

Molten bioplastic is sent to a pelletizer, such as an underwater pelletizer. The resulting pellets are suitable for use, for example, in extruders, thermoforming, injection molding, and blow moulding machines.

In some embodiments, the bioplastic resin is a thermoplastic useful for fabrication of biodegradable foams. Such a foam application does not require the addition of fibers.

In some embodiments, the biocomposite is produced with organic or inorganic particulates recovered from an organic waste stream. Thus, a high crystallinity bioplastic resin could have properties of an engineering plastic (high modulus, high strength) if it were filled with appropriate particulate material. For example, the use of 10-30% silica could lead to considerable strength enhancement. While the silica is obviously not biodegradable, it is a natural inorganic product.

### EXAMPLE 1

### Production of Biogas for the Bioplastic Resin PHB and for Biocomposites Made with Different Resins

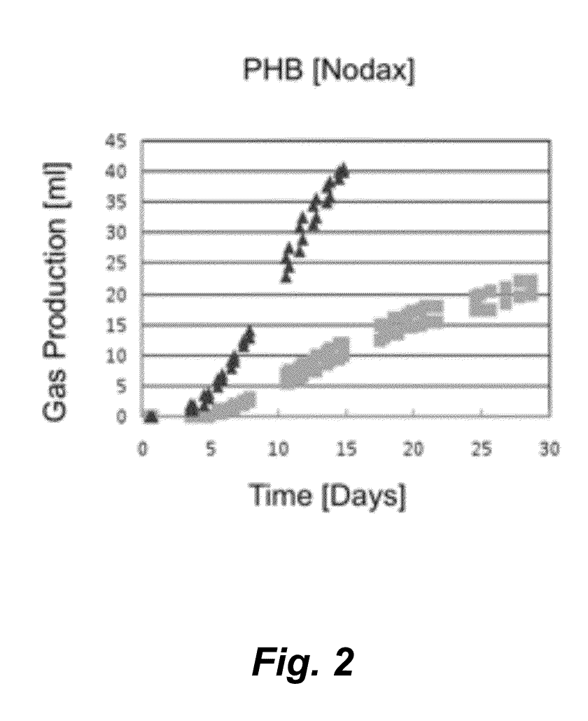

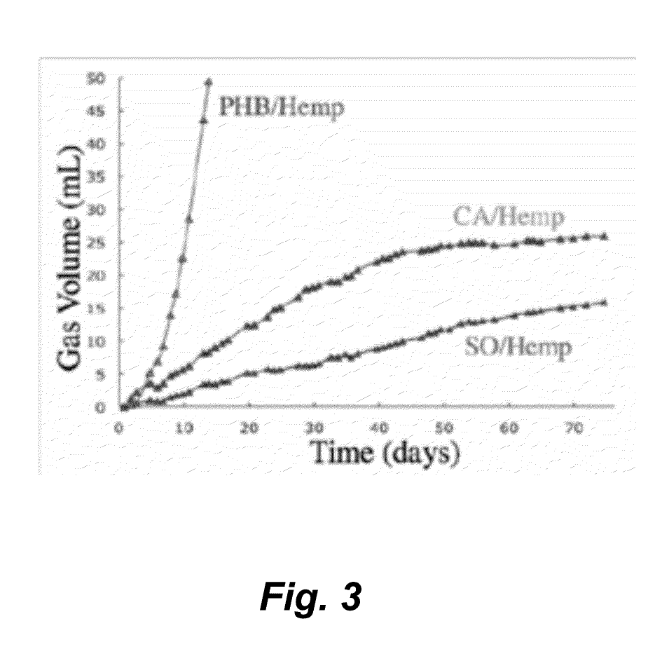

Samples of the bioplastic PHB (Nodax brand) were incubated anaerobically in microcosms containing seed material from an anaerobic digester at a wastewater treatment plant. As shown in FIG. 2, PHB degraded rapidly at 37° C. Biocomposite specimens were then produced containing PHB, cellulose acetate or soybean oil based matrix material. As shown in FIG. 3, the PHB-based biocomposites biodegraded produced biogas at a rate 8-25 times faster than their cellulose acetate and soybean oil based counterparts.

### EXAMPLE 2

### Production of the Bioplastic PHB from “Dirty” Landfill Biogas and Anaerobic Digester Biogas

Two experiments were conducted to determine the effect of biogas on the observed rate of growth and PHB production in a type II methanotroph. In the first experiment, 9 serum bottles containing 30 mL of sterilized media were inoculated with an exponential phase culture of Methylocystus parvus OBBP. Of these bottles, 3 were inoculated with 40 mL oxygen, 40 mL methane, and 40 mL CO2, to simulate uncontaminated biogas. 3 bottles were inoculated with 40 mL oxygen and 80 mL unfiltered landfill gas collected from the Palo Alto landfill, while the remaining 3 were inoculated with 40 mL oxygen and 80 mL unfiltered anaerobic digester gas collected from the San Jose wastewater treatment plant. All nine bottles were then incubated at 30 C under constant agitation, sampled periodically, and analyzed for optical density as a means of measuring total culture density.

In the second experiment, an exponential phase culture of Methylocystus parvus OBBP was centrifuged and resuspended in nitrate free media to induce PHB production. This master culture was then transferred into 9 serum bottles treated with the same gas mixtures as in experiment one. The cultures were sampled periodically, stained with Nile Red, and analyzed for fluorescence via flow cytometry to determine relative PHB concentrations. At the conclusion of the experiment, all remaining biomass was freeze-dried. The freeze-dried biomass was then analyzed for total PHB content via gas chromatography.

Growth rates for digester gas and the control gas blend were nearly identical, while growth rates for cells grown on landfill gas were substantially higher. PHB production rates were similar across the three gas types although some divergence is seen later in the PHB production period. As shown in Table 1, all three gas types resulted in significant quantities of PHB.

TABLE 1
Growth rates, doubling times, and final PHB content
for cells for cultures growing on “dirty” landfill
gas, anaerobic digester gas, and a methane/CO2 blend.

| | Growth rate | Doubling time | Final PHB |
|---|---|---|---|
| | (hours⁻¹) | (hours) | content |
| Landfill gas | 0.130 | 5.35 | 43.3 ± 15.1% |
| Digester gas | 0.099 | 7.02 | 50.7 ± 8.6% |
| Control (50% | 0.099 | 7.02 | 33.9 ± 19.5% |
| CH4/50% CO2) | | | |

### EXAMPLE 3

### Life Cycle Analysis of Biogas Feedstock

A life cycle analysis was performed for bioplastic PHB production from biogas methane. Twelve environmental impact categories were evaluated using the Building for Environmental and Economic Sustainability (BEES) 4.0 method developed by the National Institute of Standards and Technology. These categories are: Global Warming, Acidification, Eutrophication, Natural Resource Depletion, Indoor Air Quality, Habitat Alteration, Water Intake, Criteria Air Pollutants, Human Health, Smog, Ozone Depletion, and Ecological Toxicity. The study considered Cradle-to-resin production of PHB from waste biogas. Cradle-to-resin production was used as a boundary in order to easily compare the study with others that have evaluated plastic production. In addition, the Manufacture & Assembly stage and the Use & Service stage was omitted because PHAs can be processed with equipment already in use for traditional plastics and are functionally equivalent to existing petrochemical plastics during use. Results were developed on a per mass basis (functional unit: 1 kg of PHB produced) for consistent comparison with other datasets. California was used as a geographic boundary of process site.

Table 2 shows all impacts for the production of 1 kg of PHB. The values were normalized, using the BEES normalization value. A negative value is favorable. Most of the normalized values are low or negative, implying a low or net positive impact. Thus, the overall production method is favorable. The unfavorable scores were for water, acidification, human health (criteria air pollutants), ecotoxicity, smog, natural resource depletion, habitat alteration, and ozone depletion. These values they are all attributed to energy use.

TABLE 2
Impact assessment for production of 1 kg of bioplastic PHB from waste biogas (Cradle-
to-intracellular resin). Negative scores indicate benefits. The small positive
scores indicate negative effects and are largely due to energy demands.

| | | | Normalization | Normalized | Percent of |
|---|---|---|---|---|---|
| Impact Indicator | Unit | Value | Value⁶⁰ | Value | Total (%) |
| Global Warming | kg CO₂ eq | −1.94 | 6.85 × 10¹² | −2.83 × 10⁻¹³ | −0.07 |
| Acidification | H⁺ moles eq | 2.62 | 2.08 × 10¹² | 1.26 × 10⁻¹² | 0.30 |
| Carcinogenics | kg benzene eq | 1.02 × 10⁻² | 7.21 × 10 7 | 1.42 × 10⁻¹⁰ | 33.84 |
| Noncarcinogenics | kg toluene eq | 3.15 × 10¹ | 4.11 × 10¹¹ | 7.66 × 10⁻¹¹ | 18.29 |
| Respiratory Effects | kg PM2.5 eq | 1.42 × 10⁻² | 2.13 × 10¹⁰ | 6.65 × 10⁻¹³ | 0.16 |
| Eutrophication | kg N eq | 1.11 × 10⁻³ | 5.02 × 10⁹ | 2.22 × 10⁻¹³ | 0.05 |
| Ozone Depletion | kg CFC-11 eq | 4.32 × 10⁻⁷ | 8.69 × 10⁷ | 4.97 × 10⁻¹⁵ | 0.00 |
| Ecotoxicity | kg 2,4-D eq | 4.08 | 2.06 × 10¹⁰ | 1.98 × 10⁻¹⁰ | 47.29 |
| Smog | kg NOₓ eq | 1.83 × 10⁻² | 3.38 × 10¹⁰ | 5.41 × 10⁻¹³ | 0.13 |

Using biogas methane for PHB production results in a global warming potential of −1.94 kg CO₂ eq and can be as low as −2.29 if excess cell material is combusted while PHB from corn feedstock is reported to have a global warming potential of only −0.1 kg CO₂ eq.

### EXAMPLE 4

### Landfill Near Sacramento, Calif., Generates 10⁸ m³/yr of Biogas with 50% CH₄

Annual CH₄ production=5×10⁷ m³/yr=33,000 tons/yr

An energy balance indicates that use of 25% of the biogas for energy production will meet the needs for materials synthesis. This leaves ˜24,000 tons/yr for PHB production. If the PHB yield is 4 g CH₄/g PHB, the capacity for PHB production is ˜6,000 tons/yr, with no need for imported energy.

1. A process comprising:
a. Anaerobic biodegradation of one or more organic waste streams to produce biogas methane, consuming the biodegradable portion of the waste stream, and producing a refractory organic residue.
b. Use of aerobic methanotrophic bacteria to convert biogas methane into a bioplastic resin
c. Cell separation (e.g., extraction with chemicals or impingement/sonification without chemicals) and purification of the bioplastic resin
d. Renewable energy is used to meet on-site energy demands for synthesis and recovery of the bioplastic resin
e. Anaerobic biodegradation of bioproducts that contain the bioplastic resin at end-of-life so as to regenerate the biogas feedstock.

2. The process of claim 1 where the bioplastic resin is a PHA such as polyhydroxybutyrate (PHB).

3. The process of claim 1 where:
a. Renewable energy is supplied by oxidation of poorly degradable organic residue
b. Renewable energy is supplied by oxidation of a fraction of collected methane that is not used for production of the biopolymer.

4. The process of claim 1 where the bioplastic resin is a thermoplastic useful for fabrication of a biodegradable biocomposite.

5. The process of claim 4 where the biocomposite is produced with lignin and/or fibers recovered from an organic waste stream.

6. The process of claim 5 where:
a. Renewable energy is supplied by oxidation of the refractory organic residue.
b. Renewable energy is supplied by oxidation of the fraction of collected methane that is not used for production of the bioplastic resin.
c. Renewable energy is supplied by oxidation of a combination of a and b

7. The process of claim 1 where the bioplastic resin is a thermoplastic useful for fabrication of biodegradable foams.

8. The processes of claim 4 where the biocomposite is produced with organic or inorganic particulates recovered from an organic waste stream.
